(19) United States
(12) Patent Application Publication
(10) Pub. No.: US 2024/0115749 A1
(43) Pub. Date: Apr. 11, 2024

(54) Ultrasound Probe Disinfection Systems and Methods

(71) Applicant: Bard Access Systems, Inc., Salt Lake City, UT (US)

(72) Inventors: Dustin Payne, West Jordan, UT (US); William Robert McLaughlin, Bountiful, UT (US); Steffan Sowards, Salt Lake City, UT (US); Stefan Josef Fellner, Salt Lake City, UT (US)

(21) Appl. No.: 17/963,594

(22) Filed: Oct. 11, 2022

(51) Int. Cl. A61L 2/24; A61B 8/00; A61L 2/10; A61L 2/18

(52) U.S. Cl. CPC A61L 2/24; A61B 8/4254; A61B 8/4405; A61B 8/4444; A61L 2/10; A61L 2/186; A61L 2202/11; A61L 2202/122; A61L 2202/14; A61L 2202/24

(57) ABSTRACT

System and methods for disinfecting ultrasound probes including a disinfection module operably and physically coupled with an ultrasound-imaging system including an ultrasound probe. The disinfection module is configured to expose the external surface of the ultrasound probe to UV light to define a high-level disinfection process. Sensors determine when the ultrasound probe is disposed within a disinfecting cavity of the disinfection module and logic governs the operation of UV light sources. Logic determines a contamination level of the probe and defines an appropriate disinfection level. Exposing the probe to hydrogen peroxide is an alternative to the UV light. Logic may activate the high-level disinfection process upon placement of the probe within the cavity.

### BACKGROUND

The application of ultrasound imaging during medical procedures continues to expand. Consequently, the risk of a patient contracting a disease or infection via the ultrasound imaging procedure is increased as well. As ultrasound probes are in direct contact with a patient, including body fluids such as blood, disinfecting the ultrasound probe is a key aspect in preventing transmission of viruses, bacteria, or other forms of contamination between patients. Disinfection processes for ultrasound probes may be manually performed clinicians and may require a significant time period, resulting in labor costs and downtime for the ultrasound probe.

Disclosed herein are systems and methods for automatically applying a high-level disinfection process to an ultrasound probe that address the forgoing.

### SUMMARY

Disclosed herein is an ultrasound-imaging system that, according to some embodiments, includes (i) an ultrasound probe having a probe body extending between a proximal end and a distal end, where the probe body defines an external surface; (ii) a disinfection module configured to apply a high-level disinfection process to at least a portion of the external surface, where the disinfection module includes a disinfection housing defining a cavity configured to receive the ultrasound probe therein; and (iii) a system module including a display and a console coupled with the ultrasound probe and the disinfection module, where the console includes one or more processors and a non-transitory computer-readable medium having stored thereon logic that, when executed by the one or more processors, causes operations that include activating the high-level disinfection process to disinfect the at least a portion of the external surface.

In some embodiments, the disinfection module and the system module are attached to a rolling stand.

In some embodiments, the disinfection housing is at least one of (i) directly coupled with or (ii) incorporated into a housing of the system module.

In some embodiments, the disinfection housing includes a drawer defining the cavity.

In some embodiments, the cavity includes a slot extending downward from a top end of the disinfection housing, the slot configured to receive the ultrasound probe such that (i) the at least a portion of the external surface is disposed within the cavity, and (ii) the distal end of the probe body is directed toward a bottom end of the disinfection housing.

In some embodiments, at least one of the disinfection module or ultrasound probe includes a presence sensor configured to detect the presence of the ultrasound probe within the cavity, and the operations include activating the high-level disinfection process in response to a signal from the presence sensor.

In some embodiments, the cavity is configured to orient the ultrasound probe such that the distal end of the probe body is directed vertically upward, and in some embodiments, the ultrasound probe includes an orientation sensor, including at least one of a gyroscope or an accelerometer, configured to determine when the distal end of the probe body is directed vertically upward. In such embodiments, the operations include activating the high-level disinfection process in response to a signal from the orientation sensor.

In some embodiments, the operations include activating the high-level disinfection process upon receiving input from a clinician including one or more of pushing a button, touching a user interface screen, or issuing a voice command.

In some embodiments, the operations include comparing a usage parameter of the ultrasound probe with a usage threshold stored in the non-transitory computer-readable medium. As a result of the comparison, the operations may include (i) applying a first disinfection level when the usage parameter is less than the threshold, or (ii) applying a second disinfection level when the usage parameter exceeds the threshold, where the second disinfection level is greater than the first disinfection level.

In some embodiments, applying the high-level disinfection process includes exposing the at least a portion of the external surface to an ultraviolet light having a wavelength between about 100 nm and 400 nm. In some embodiments, applying the high-level disinfection process includes exposing the at least a portion of the external surface to hydrogen peroxide.

Also disclosed herein is an ultrasound probe that, according to some embodiments, includes a probe body extending between a proximal end and a distal end, the probe body defining an external surface, where the ultrasound probe is configured for disinfection of at least a portion of the external surface via a high-level disinfection process when the ultrasound probe is disposed within a cavity of a disinfection module, and where the ultrasound probe and the disinfection module are coupleable with an ultrasound-imaging system module.

In some embodiments of the probe, at least one of the ultrasound probe or the disinfection module includes a presence sensor. In such embodiments, the ultrasound probe is configured to activate the presence sensor based on the presence of the ultrasound probe within the cavity, and the ultrasound system module is configured to activate the high-level disinfection process based on a signal from the presence sensor.

In some embodiments of the probe, the cavity is configured to orient the ultrasound probe such that the distal end of the probe body is directed vertically upward, and the ultrasound probe includes an orientation sensor including at least one of a gyroscope or an accelerometer configured to determine when the distal end of the probe body is directed vertically upward. In such embodiments, the ultrasound-imaging system module is configured to activate the high-level disinfection process based on a signal from the orientation sensor.

Also disclosed herein is a method a disinfecting an ultrasound probe that, according to some embodiments, includes (i) detecting a presence of the ultrasound probe within a cavity of a disinfection module, where the ultrasound probe includes probe body defining an external surface extending between a proximal end and a distal end of the ultrasound probe; and (ii) applying a high-level disinfection process to at least a portion of an external surface of a body of the ultrasound probe when the ultrasound probe is disposed within the cavity.

In some embodiments of the method, applying the high-level disinfection process includes exposing the at least a portion of the external surface to at least one of an ultraviolet light having a wavelength between about 100 nm and 400 nm or hydrogen peroxide.

In some embodiments, applying the high-level disinfection process includes activating the high-level disinfection process in response to one or more of a pushing of a button, a touching of a user interface screen, or an issuing of a voice command.

In some embodiments of the method, at least one of the disinfection module or the ultrasound probe includes a presence sensor configured to detect the presence of the ultrasound probe within the cavity, where applying the high-level disinfection process includes activating the high-level disinfection process based on a signal from the presence sensor.

In some embodiments of the method, the cavity is configured to orient the ultrasound probe such that the distal end of the probe body is directed vertically upward, and the ultrasound probe includes an orientation sensor, including at least one of a gyroscope or an accelerometer, configured to determine when the distal end of the probe body is directed vertically upward. In such embodiments, applying the high-level disinfection process includes activating the high-level disinfection process based on a signal from the orientation sensor.

In some embodiments of the method, applying a high-level disinfection process includes comparing a usage parameter of the ultrasound probe with a usage threshold. As a result of the comparison, the applying the high-level disinfection process may include (i) applying a first disinfection level when the usage parameter is less than the threshold or (ii) applying a second disinfection level when the usage parameter exceeds the threshold, where the second disinfection level is greater than the first disinfection level.

These and other features of the concepts provided herein will become more apparent to those of skill in the art in view of the accompanying drawings and following description, which describe particular embodiments of such concepts in greater detail.

### DRAWINGS

FIG. 1 illustrates an ultrasound-imaging system, in accordance with some embodiments.

FIG. 2 illustrates a block diagram of a console of the ultrasound-imaging system of FIG. 1 in accordance with some embodiments.

FIG. 3 illustrates a portion of another embodiment of the ultrasound-imaging system, in accordance with some embodiments.

FIG. 4 illustrates another embodiment of the disinfection module of the system of FIG. 1, in accordance with some embodiments.

FIG. 5 is a flow chart of the disinfection method of an ultrasound probe, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The terms “proximal” and “distal” refer to opposite ends of a medical device, including the devices disclosed herein. As used herein, the proximal portion of a medical device is the portion nearest a practitioner during use, while the distal portion is the portion at the opposite end. For example, the proximal end of an ultrasound probe is defined as the end closest to the practitioner during utilization of the ultrasound probe. The distal end is the end opposite the proximal end, along the longitudinal direction of the ultrasound probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art. References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

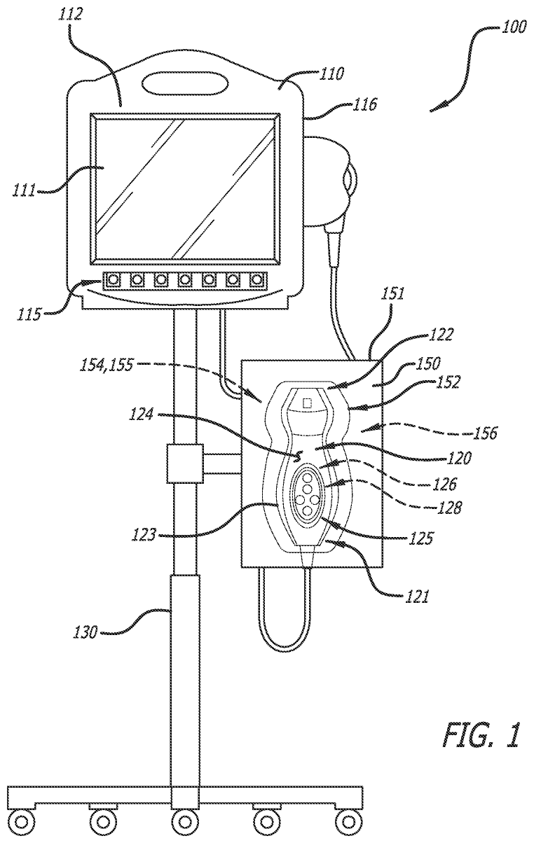

FIG. 1 illustrates an ultrasound-imaging system (system) 100 generally configured to obtain ultrasound images from a patient and depict the ultrasound images on a display 111 of a system module 110. The system module 110 may include a number of input devices 115, such as buttons, dials, and the like configured to receive input from a clinician. Similarly, the display 111 may include a graphic user interface (GUI) including a touch screen configured to receive input from the clinician. The system module 110 includes console 112 disposed within a system module housing 116. The system module 110 may be attached to a support structure 130, which, in some embodiments, may include a rolling stand as illustrated.

The system 100 generally includes an ultrasound probe (probe) 120 operative coupled with the system module 110. The probe 120 includes a probe body 123 extending between a proximal end 121 and a distal end 122 of the probe 120. The probe body 123 defines an external surface 124. Similar to the system module 110, the probe 120 may include number of input devices 125, such as buttons, dials, and the like configured to receive input from the clinician. During use, the external surface 124 may become contaminated, such as via patient or clinician contact. As such, the probe 120 may need to be disinfected between uses of the probe 120.

The system further includes a disinfection module 150 coupled with the system module 115. The disinfection module 150 is generally configured to apply a high-level disinfection process to the ultrasound probe 120. More specifically, the disinfection module 150 is configured to apply the high-level disinfection process to the external surface 124 of the probe body 123. In some embodiments, the disinfection module 150 may be configured to apply the high-level disinfection process to only a portion of the external surface 124, such as a distal portion of the external surface 124, where the distal portion may be the portion of the external surface 124 that is generally in contact with the patient during use. The distal portion may include the distal end 122 and may further include portions of the external surface extending proximally away from the distal end 122.

The disinfection module 150 is communicatively coupled with the console 112, such as via a cable, for example. In the illustrated embodiments, the disinfection module 150 receives electrical power from the console 112 and is operatively coupled with the console 112 so that logic of the console 112 governs operation of the disinfection module 150. Alternatively, the disinfection module 150 may be wirelessly coupled with the console 112. In such alternative embodiments, the disinfection module 150 may include a separate console in communication with the console 112. Such an alternative embodiment may be advantageous when the disinfection module 150 is added to an existing ultrasound-imaging system 100.

Similar to the system module 110, the disinfection module 150 may be attached to the support structure 130. In some embodiments, the housing 151 may be directly attached to the system module housing 116. In some embodiments, the housing 151 may be incorporated into the system module housing 116. In some embodiments, the disinfection module 150 may be attached to the support structure 130 so that the housing 151 (including the cavity 152) is disposed in a defined orientation. In some embodiments, the cavity 152 may be vertically oriented so that the ultrasound probe 120 is vertically oriented when disposed within the cavity 152. More specifically, the cavity 152 may cause the ultrasound probe 120 to be vertically oriented so that the distal end 122 is directed vertically upward.

The disinfection module 150 includes a housing 151 that defines a cavity 152 configured to receive the ultrasound probe 120 therein. The disinfection module 150 is generally configured to apply the high-level disinfection process to the ultrasound probe 120 when the ultrasound probe 120 is disposed within the cavity 152. The cavity 152 may also define a storage location for the ultrasound probe 120. In some embodiments, the housing 151 may entirely enclose the ultrasound probe 120 when the ultrasound probe 120 is disposed within the cavity 152.

The disinfection module 150 may, according to some embodiments, include a number (e.g., 1, 2, or more) of presence sensors 156 configured to detect the presence of the ultrasound probe 120 within the cavity 152, and thereby provide a presence signal to the console 112 indicating that the ultrasound probe 120 is disposed within the cavity 112. The presence sensors 156 may include any suitable sensor configured to detect the presence of the ultrasound probe 120, such as a switch, a proximity sensor, a capacitance sensor, an inductive sensor, an optical sensor or a hall effect sensor, for example. Similar to the presence sensors 156 of the disinfection module 150, the probe 120 may include as an alternative or in addition to the presence sensors 156, a number (e.g., 1, 2, or more) of probe presence sensors 126 configured to detect the presence of the ultrasound probe 120 within the cavity 152, and thereby provide a presence signal to the console 112 indicating that the ultrasound probe 120 is disposed within the cavity 112.

In some embodiments, the probe 120 may include a number (e.g., 1, 2, or more) of probe orientation sensors 128 configured to determine an orientation of the probe 120, such as a gyroscope or an accelerometer, for example. By way of example, the orientation sensors 128 may determine if the probe 120 is oriented so that the distal end 122 is directed vertically upward as may be consistent with disposition of the probe 120 with the cavity 152. The orientation sensors 128 may also determine if the probe 120 is oriented so that the distal end 122 is directed generally downward (or otherwise not directed vertically upward) as may be consistent with use of the probe 120 in obtaining ultrasound images. By way of summary, the probe orientation sensors 128 may provide one orientation signal that indicates that the probe 120 is disposed within the cavity 152 and another orientation signal that indicates that the probe 120 is in use.

In the illustrated embodiment, the disinfection module 150 include a number (e.g., 1, 2, 3, 4, 5, 6, or more) of ultraviolet light sources 154. The ultraviolet (UV) light sources 154 are positioned and arranged about the cavity 152 within the housing 151 so as to project UV light onto the external surface 124, or portion thereof, when the probe 120 is disposed within the cavity 152. Studies have shown that UV light can be effective in performing a high-level disinfection process on reusable medical surfaces, such as the external surface 124 of the reusable probe 120. More specifically, the UV light within the UV-C wavelength range (i.e., 100-280 nm) has been shown to be advantageous in performing high-level disinfection. In some embodiments, the ultraviolet light sources 154 may project UV light having a wavelength between about 100 nm and 400 nm. In some embodiments, the disinfection module 150 may provide for the projection of different UV light intensities. The different intensities may be defined by activating different subsets of the UV light sources 154 or by activating individual UV light sources 154 at different excitation levels. As such, the disinfection module 150 may be configured to perform different levels of disinfection. The ultraviolet light sources 154 are coupled with the console 112 so that logic of the console 112 may govern the activation of the UV light sources 154.

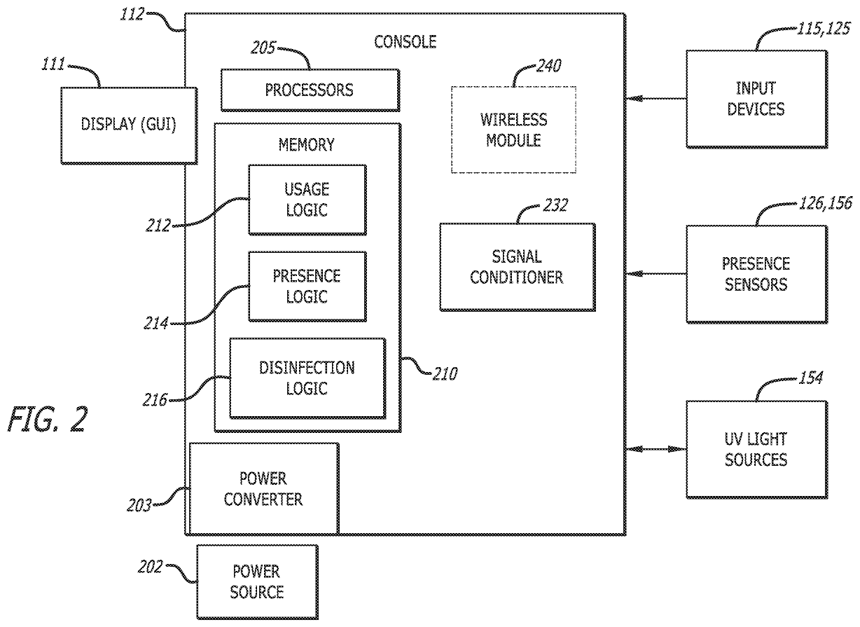

FIG. 2 illustrates a block diagram of the console 112 of the ultrasound-imaging system 100 in accordance with some embodiments. The console 112 receives power from an external power source 202, and a power converter 203 defines and distributes electrical power to the other console components. The console 112 includes a number of processors 205 coupled with memory 210 including a non-transitory computer-readable medium. Logic stored in the memory 210 includes usage logic 212, presence logic 214, and disinfection logic 216. The presence sensors 126, 156 are couple with the console 112 and receive power from the console 112 via the power converter 203, and a signal conditioner 232 receives and converts sensor signals into sensor data for processing by the presence logic 214. The UV light sources 154 are coupled with the console 112 and receive power from the power converter 203.

In some embodiments, the console 112 may include a wireless module 240. The wireless module 240 may facilitate communication between the system module 110 and the disinfection module 150, according to some embodiments. The wireless module 240 may also facilitate communication between the system module 110 and external computing devices such as a cell phone, a tablet, or an electronic medical record system, according to some embodiments.

The disinfection logic 216 generally governs the operation of the UV light sources 154. In some embodiments, the disinfection logic 216 may govern the operation of the UV light sources 154 in accordance with usage data received from the usage logic 212 and/or presence data received from the presence logic 214. For example, the disinfection logic 216 may only activate the UV light sources 154 when the data from the presence logic 214 indicates that the probe 120 is disposed within the cavity 152. In some embodiments, the disinfection logic 216 may activate (i.e., turn “on” and/or “off”) the UV light sources 154 in accordance with (i.e., based on) an input signal from one or more of the input devices 115, the probe input devices 125, or the GUI.

In some embodiments, the disinfection logic 216 maintain activation of the UV light sources 154 for a defined duration. For example, in some embodiments, the disinfection logic 216 may maintain activation of the UV light sources 154 for a first duration or a second duration, where the second duration is different from the first duration. In some embodiments, the UV light sources 154 may be configured to project UV light at different intensities. As such, in some embodiments, the disinfection logic 216 may activate (i.e., energize or excite) the UV light sources 154 at a first intensity or a second intensity, where the second intensity is different from the first intensity. By way of summary, the disinfection logic 216 may be configured to apply a first disinfection level and a second disinfection level, where the second disinfection level is different from the first disinfection level.

In some embodiments, as an alternative to exposing the external surface 124 to UV light to disinfect the external surface 124, the disinfection module 150 may be configured to expose the external surface 124 to a hydrogen peroxide solution to chemically disinfect the external surface 124. As such, the disinfection module 150 may include a hydrogen peroxide application mechanism 155 as an alternative to the UV light sources 154. The application mechanism 155 may include hydraulic components, such as a container, tubing, pumps, valves, and/or spray nozzles and the like, to enable to the disinfection module 150 to apply the hydrogen peroxide solution to the external surface 124. The application mechanism 155 may be coupled with the console 112 so that the disinfection logic 216 logic may govern the operation of the application mechanism 155.

The presence logic 214 is configured to receive presence signals from the presence sensors, i.e., the presence sensors 156 and/or the probe presence sensors 126, and provide the presence data to the disinfection logic 216 so that the disinfection logic 216 may activate the high level disinfection process based on presence signals from the presence sensors 156 and/or the probe presence sensors 126. For example, the disinfection logic 216 may refrain from activating the high level disinfection process unless the presence signals indicate that the probe 120 is disposed in the cavity 152. In some embodiments, the disinfection logic 216 may automatically activate the high level disinfection process in direct response to a presence signal indicating that the probe 120 is disposed in the cavity 152.

The presence logic 214 may also be configured to receive orientation signals from the orientation sensors 128, and provide the orientation data to the disinfection logic 216 so that the disinfection logic 216 may activate the high level disinfection process based on orientation signals from the orientation sensors 128. For example, the disinfection logic 216 may refrain from activating the high level disinfection process unless the orientation signals indicate that the probe 120 is oriented such the distal end 122 is directed vertically upward consistent with the probe 120 disposed in the cavity 152. In some embodiments, the disinfection logic 216 may automatically activate the high level disinfection process in direct response to an orientation signal indicating that the probe 120 is oriented such the distal end 122 is directed vertically upward consistent with the probe 120 disposed in the cavity 152.

In some embodiments, the presence logic 214 may determine a present time period, i.e., a time period for which the probe 120 has been continuously disposed within the cavity 152. Similarly, the presence logic 214 may determine a non-present time period, i.e., a time period for which the probe 120 has been continuously removed from (i.e., not present within) the cavity 152. In some embodiments, the disinfection logic 216 may utilize the non-present time period to determine the UV light duration or intensity (i.e., disinfection level).

The usage logic 212 may determine different levels of use of the probe 120. For example, the usage logic 212 may determine a duration of the use of the probe 120, such as an energized duration of the probe 120. In some instances, the duration of the use of the probe 120 may align with a level of contamination of the probe 120, and the level of contamination of the probe 120 may, in turn, define a needed disinfection level of the probe 120. For example, low level of use of the probe 120 (e.g., a short duration of use) may indicate a low level of contamination requiring a decreased level of disinfection. Similarly, a high level of use of the probe 120 (e.g., a long duration of use) may indicate a high level of contamination requiring an increased level of disinfection.

In some embodiments, the usage logic 212 may compare a usage parameter with a usage threshold stored in memory (e.g., the non-transitory computer-readable medium) where the usage parameter is indicative of a contamination level of the probe 120. As a result of the comparison, the usage data may include a first disinfection level needed to disinfect the probe 120 when the usage parameter is less than the usage threshold. Alternatively, the usage data may include a second disinfection level needed to disinfect the probe 120 when the usage parameter exceeds the usage threshold, where the second disinfection level is greater than the first disinfection level.

In some embodiments, the usage logic 212 may receive input from the clinician as to a contamination level of the probe 120. For example, the clinician may provide input to the system 100 that the probe 120 was used in accordance with a high contamination risk procedure, such as direct exposure to blood, or use with a high risk patient, for example. In such an instance, the usage logic 212 may include the second disinfection level.

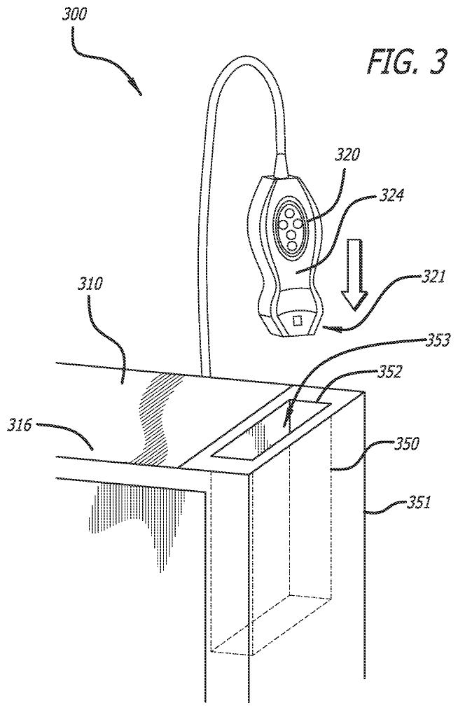

FIG. 3 illustrates another embodiment of an ultrasound-imaging system 300 that can, in certain respects, resemble components of the ultrasound-imaging system 100 described in connection with FIGS. 1-2. It will be appreciated that all the illustrated embodiments may have analogous features. Accordingly, like features are designated with like reference numerals. Relevant disclosure set forth above regarding similarly identified features thus may not be repeated hereafter. Moreover, specific features of the ultrasound-imaging system 100 and related components shown in FIGS. 1-2 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the ultrasound-imaging system 300 of FIG. 3. Any suitable combination of the features, and variations of the same, described with respect to the ultrasound-imaging system 100 and components illustrated in FIGS. 1-2 can be employed with the ultrasound-imaging system 300 and components of FIG. 3, and vice versa. This pattern of disclosure applies equally to further embodiments depicted in subsequent figures and described hereafter.

FIG. 3 is a perspective view of a portion the ultrasound-imaging system (system) 300 including the ultrasound probe 320. The system 300 includes a system module 310 and a disinfection module 350. The housing 351 of the disinfection module 350 is incorporated into the system module housing 316. The cavity 352 includes a slot 353 extending downward from a top side of the system module housing 316. The cavity 352 is configured to receive a distal portion of the probe 320 when the probe 320 is inserted into the slot 353. The disinfection module 350 is configured to apply the high-level disinfection process to a distal portion of the external surface 324, such as a distal portion of the external surface 324, where the distal portion includes the distal end 322 and portions of the external surface extending proximally away from the distal end 322.

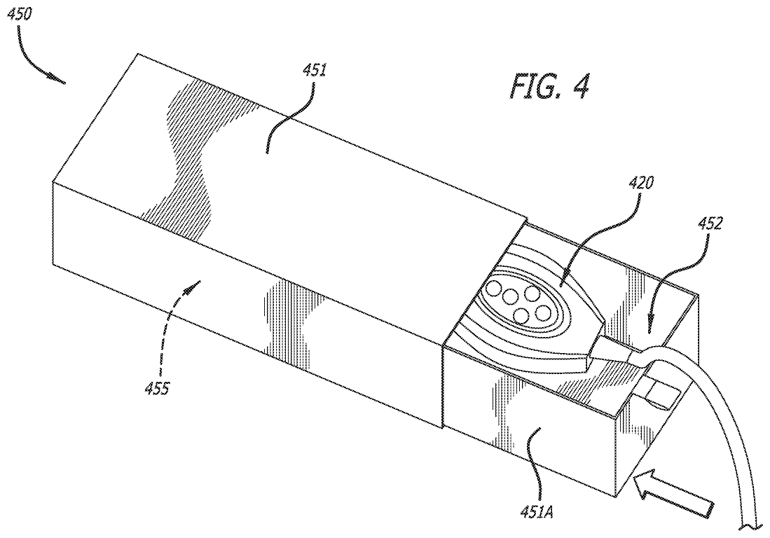

FIG. 4 illustrates another embodiment of a disinfection module 450 that can, in certain respects, resemble components and functionality of the disinfection module 150 described in connection with FIGS. 1-2. The disinfection module 450 includes a housing 451 and housing drawer 451A configured for slidable displacement into and out of the housing 451. The housing drawer 451A defines the cavity 452 configured to receive the ultrasound probe 420 such that when the housing drawer 451A is fully closed, the ultrasound probe 420 is fully enclosed by the housing 451. The disinfection module 450 may include a drawer sensor 455 configured to determine when the housing drawer 451A is fully closed and provide a drawer status signal to the logic (e.g., the presence logic 214) indicating that the high-level disinfection process may be activated.

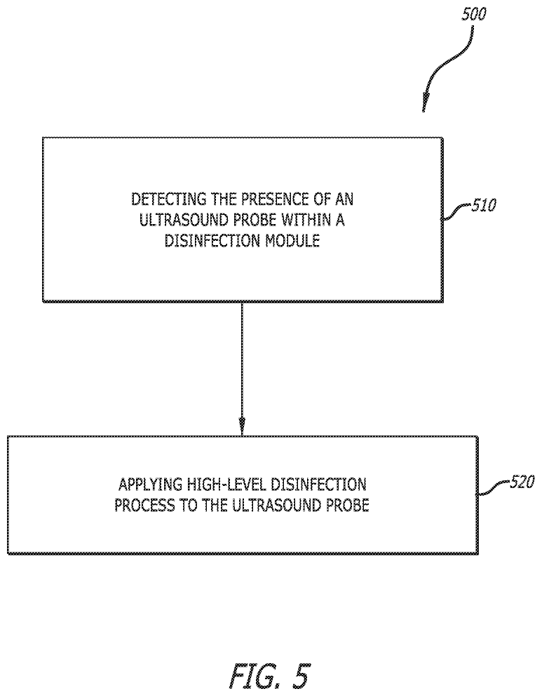

FIG. 5 is a flow chart illustrating an exemplary disinfection method of the ultrasound probe that, according to some embodiments, may include all or a subset of the following steps or processes. The method 500 includes detecting the presence of the ultrasound probe within the cavity of the disinfection module (block 510). During use, the clinician may place the ultrasound probe within the cavity. Thereafter, the presence sensor may detect the presence of the ultrasound probe within the cavity. In some embodiments, detecting the presence of the ultrasound probe within the cavity may include determining that the ultrasound probe is oriented so that the distal end of the ultrasound probe is directed vertically upward. In some embodiments, detecting the presence of the ultrasound probe within the cavity may include determining that a drawer of the disinfection module is fully closed.

The method 500 further includes applying the high-level disinfection process to at least a portion of the external surface of the ultrasound probe (block 520) when the ultrasound probe is disposed within a cavity of the disinfection module. In some embodiments, applying the high-level disinfection process includes exposing the at least a portion of the external surface of the ultrasound probe to an ultraviolet light having a wavelength between about 100 nm and 400 nm. In some embodiments, applying the high-level disinfection process includes exposing the at least a portion of the external surface of the ultrasound probe to a hydrogen peroxide solution.

In some embodiments, applying the high-level disinfection process includes activating the high-level disinfection process in response a pushing of a button, a touching of a user interface screen, or an issuing of a voice command. In some embodiments, applying the high-level disinfection process includes activating the high-level disinfection process based on receiving the presence signal from the presence sensor that the ultrasound probe is within the cavity. In some embodiments, applying the high-level disinfection process includes automatically activating the high-level disinfection process in direct response to receiving the signal from the presence sensor that the ultrasound probe is within the cavity

In some embodiments, applying the high-level disinfection process includes activating the high-level disinfection process based on an orientation signal from an orientation sensor of the ultrasound probe that the distal end of the ultrasound probe is directed vertically upward.

In some embodiments, applying the high-level disinfection process includes comparing a usage parameter of the ultrasound probe with a usage threshold. As a result of the comparison, applying the high-level disinfection process may include (i) applying a first disinfection level when the usage parameter is less than the threshold, or (ii) applying a second disinfection level when the usage parameter exceeds the threshold, where the second disinfection level is greater than the first disinfection level.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An ultrasound-imaging system, comprising:
an ultrasound probe having a probe body extending between a proximal end and a distal end, the probe body defining an external surface;
a disinfection module configured to apply a high-level disinfection process to at least a portion of the external surface, the disinfection module including a disinfection housing defining a cavity configured to receive the ultrasound probe therein; and
a system module, comprising:
a display; and
a console coupled with the ultrasound probe and the disinfection module, the console including one or more processors and a non-transitory computer-readable medium having stored thereon logic that, when executed by the one or more processors, causes operations, including activating the high-level disinfection process to disinfect the at least a portion of the external surface.

2. The system of claim 1, wherein the disinfection module and the system module are attached to a rolling stand.

3. The system of claim 1, wherein the disinfection housing is at least one of (i) directly coupled with or (ii) incorporated into a housing of the system module.

4. The system of claim 3, wherein the cavity includes a slot extending downward from a top end of the disinfection housing, the slot configured to receive the ultrasound probe therein such that the at least a portion of the external surface is disposed within the cavity, and the distal end of the probe body is directed toward a bottom end of the disinfection housing.

5. The system of claim 1, wherein disinfection housing the includes a drawer defining the cavity.

6. The system of claim 1, wherein:
at least one of the disinfection module or the ultrasound probe includes a presence sensor configured to detect the presence of the ultrasound probe within the cavity, and
the operations include activating the high-level disinfection process based a signal from the presence sensor.

7. The system of claim 1, wherein the cavity is configured to orient the ultrasound probe such that the distal end of the probe body is directed vertically upward.

8. The system of claim 7, wherein:
the ultrasound probe includes an orientation sensor, including at least one of a gyroscope or an accelerometer, configured to determine when the distal end of the probe body is directed vertically upward, and
the operations include activating the high-level disinfection process based on a signal from the orientation sensor.

9. The system of claim 1, wherein the operations include activating the high-level disinfection process upon receiving input from a clinician, the input including one or more of a pushing of a button, a touching of a user interface screen, or an issuing of a voice command.

10. The system of claim 1, wherein the operations further include:
comparing a usage parameter of the ultrasound probe with a usage threshold stored in the non-transitory computer-readable medium; and
as a result of the comparison, activating the high-level disinfection process according to (i) a first disinfection level when the usage parameter is less than the threshold or (ii) a second disinfection level when the usage parameter exceeds the threshold, the second disinfection level greater than the first disinfection level.

11. The system of claim 1, wherein activating the high-level disinfection process includes exposing the at least a portion of the external surface to an ultraviolet light having a wavelength between about 100 nm and 400 nm.

12. The system of claim 1, wherein the operations further include activating the high-level disinfection process includes exposing the at least a portion of the external surface to a hydrogen peroxide solution.

13. An ultrasound probe, comprising:
a probe body extending between a proximal end and a distal end, the probe body defining an external surface,
wherein:
the ultrasound probe is configured for disinfection of at least a portion of the external surface via a high-level disinfection process when the ultrasound probe is disposed within a cavity of a disinfection module, and
the ultrasound probe and the disinfection module are coupleable with an ultrasound system module.

14. The ultrasound probe of claim 13, wherein:
at least one of the ultrasound probe or the disinfection module includes a presence sensor,
the ultrasound probe is configured to activate the presence sensor based on the presence of the ultrasound probe within the cavity, and
the ultrasound system module is configured to activate the high-level disinfection process based on a signal from the presence sensor.

15. The ultrasound probe of claim 13, wherein:
the cavity is configured to orient the ultrasound probe such that the distal end of the probe body is directed vertically upward,
the ultrasound probe includes an orientation sensor, including at least one of a gyroscope or an accelerometer, configured to determine when the distal end of the probe body is directed vertically upward, and
the ultrasound system module is configured to activate the high-level disinfection process based on a signal from the orientation sensor.

16. A method of disinfecting an ultrasound probe, comprising:
detecting a presence of the ultrasound probe within a cavity of a disinfection module, the ultrasound probe including probe body defining an external surface extending between a proximal end and a distal end of the ultrasound probe; and
applying a high-level disinfection process to at least a portion of the external surface when the ultrasound probe is disposed within the cavity of a disinfection module.

17. The method of claim 16, wherein applying the high-level disinfection process includes exposing the at least a portion of the external surface to at least one of (i) an ultraviolet light having a wavelength between about 100 nm and 400 nm or (ii) a hydrogen peroxide solution.

18. The method of claim 16, wherein applying a high-level disinfection process includes activating the high-level disinfection process based on input from a clinician, the input including one or more of a pushing of a button, a touching of a user interface screen, or an issuing of a voice command.

19. The method of claim 16, wherein:
at least one of the disinfection module or ultrasound probe includes a presence sensor configured to detect a presence of the ultrasound probe within the cavity, and
applying a high-level disinfection process includes activating the high-level disinfection process based on a signal from the presence sensor.

20. The method of claim 16, wherein:
the cavity is configured to orient the ultrasound probe such that the distal end of the probe body is directed vertically upward,
the ultrasound probe includes an orientation sensor including at least one of a gyroscope or an accelerometer configured to determine when the distal end of the probe body is directed vertically upward, and
applying a high-level disinfection process includes activating the high-level disinfection process based on a signal from the orientation sensor.

21. The method of claim 16, wherein applying a high-level disinfection process includes:
comparing a usage parameter of the ultrasound probe with a usage threshold; and
as a result of the comparison, (i) applying a first disinfection level when the usage parameter is less than the threshold, or (ii) applying a second disinfection level when the usage parameter exceeds the threshold, the second disinfection level greater than the first disinfection level.
